(19) United States
(12) Patent Application Publication
(10) Pub. No.: US 2018/0038830 A1
(43) Pub. Date: Feb. 8, 2018

(54) NANOPORE-BASED DNA SENSING DEVICE WITH NEGATIVE CAPACITANCE FOR IMPROVED DNA SENSING SIGNAL

(71) Applicant: QUALCOMM Incorporated, San Diego, CA (US)

(72) Inventors: Yong Ju LEE, San Diego, CA (US); Joung Won PARK, San Diego, CA (US); Vladimir APARIN, San Diego, CA (US)

(21) Appl. No.: 15/226,859

(22) Filed: Aug. 2, 2016

(51) Int. Cl. G01N 27/447; C12Q 1/68

(52) U.S. Cl. CPC G01N 27/44791; C12Q 1/6869; H01L 23/5226

(57) ABSTRACT

Techniques for improving the DNA sensing signal of nanopore-based DNA sensing devices are disclosed. A related DNA sensing device may include a first electrode, a second electrode, a hydrophobic layer having a nanopore disposed therein, and a negative capacitance layer.

### INTRODUCTION

Aspects of this disclosure relate generally to sensing devices for deoxyribonucleic acid (DNA), and more particularly to methods and apparatuses for improving the DNA sensing signal of nanopore-based DNA sensing devices.

DNA, sometimes referred to as the “blueprint of life”, is a molecule that stores biological information. The structure of DNA, famously discovered by James Watson and Francis Crick, consists of two strands of biopolymer, coiled around one another to form a double helix. Each strand is a polynucleotide that includes a plurality of nucleotides, for example, cytosine (“C”), guanine (“G”), adenine (“A”), and thymine (“T”). Each nucleotide in a first strand of DNA may be bonded to a paired nucleotide in the second strand, thereby forming a base pair. Generally, cytosine and guanine are paired to form a “G-C” or “C-G” base pair, and adenine and thymine are paired to form an “A-T” or “T-A” base pair.

Although the structure of DNA is now known, new methods for analyzing individual DNA molecules are still being developed. Generally, the analysis includes ‘reading’ the nucleotide sequence of a particular DNA strand. In one method, known as nanopore DNA sequencing, a nanopore is immersed in a conductive fluid, and a voltage is applied across the nanopore. As a result, ions are conducted through the nanopore, thereby generating a measurable electric current. A DNA strand is then transmitted through a nanopore, one nucleotide at a time. The presence of a nucleotide within the nanopore disrupts the conduction of the ions, thereby causing a change in the electric current. Moreover, the change in electrical current due to a particular nucleotide differs from the change in electrical current due to other nucleotides. Accordingly, an entire DNA strand can be transmitted through the nanopore and each nucleotide in the strand can be identified based on the change in current. Over time, the changes in electric current result in a DNA sensing signal reflecting the particular nucleotides in a particular DNA strand.

As nanopore DNA sequencing improves, new challenges are presented. For example, a capacitance may arise within the DNA sensing device. The capacitance may limit the bandwidth of the DNA sensing signal by reducing the maximum cutoff frequency. The capacitance may also increase a noise component of the DNA sensing signal. As a result, new technologies are needed for improving the DNA sensing signal of nanopore-based DNA sensing devices.

### SUMMARY

Techniques for increasing the lifetime of nanopore-based DNA sensing devices are disclosed.

In one example, a DNA sensing device is disclosed. The DNA sensing device may include, for example, a first electrode, a second electrode, a hydrophobic layer having a nanopore disposed therein, and a negative capacitance layer.

In another example, a method of fabricating a DNA sensing device is disclosed. The method of fabricating a DNA sensing device may include, for example, providing a first electrode, providing a second electrode, providing a hydrophobic layer, disposing a nanopore in the hydrophobic layer, and providing a negative capacitance layer.

### BRIEF DESCRIPTION OF THE DRAWINGS

The accompanying drawings are presented to aid in the description of various aspects of the disclosure and are provided solely for illustration of the aspects and not limitation thereof.

FIG. 1 generally illustrates a DNA sensor array in accordance with aspects of the disclosure.

FIG. 2 generally illustrates a nanopore-based DNA sensing device in accordance with an aspect of the disclosure.

FIG. 3 generally illustrates the separation and/or combination of a double-stranded DNA molecule.

FIG. 4A generally illustrates a circuitry model associated with the nanopore-based DNA sensing device of FIG. 2.

FIG. 4B generally illustrates a circuitry model with negative capacitance in accordance with aspects of the disclosure.

FIG. 5 generally illustrates a DNA sensing device with a negative capacitor in accordance with aspects of the disclosure.

FIG. 6A generally illustrates a negative capacitor in accordance with aspects of the disclosure.

FIG. 6B generally illustrates a negative capacitor in accordance with other aspects of the disclosure.

FIG. 6C generally illustrates a negative capacitor in accordance with yet other aspects of the disclosure.

FIG. 7 generally illustrates a method for fabricating the DNA sensing device of FIG. 5.

FIG. 8 generally illustrates another DNA sensing device with a negative capacitor in accordance with other aspects of the disclosure.

FIG. 9A generally illustrates a negative capacitor in accordance with yet other aspects of the disclosure.

FIG. 9B generally illustrates a negative capacitor in accordance with yet other aspects of the disclosure.

FIG. 9C generally illustrates a negative capacitor in accordance with yet other aspects of the disclosure.

FIG. 10 generally illustrates a method for fabricating the DNA sensing device of FIG. 8.

FIG. 11A generally illustrates the DNA sensing device of FIG. 8 in a first stage of fabrication.

FIG. 11B generally illustrates the DNA sensing device of FIG. 8 in a second stage of fabrication.

FIG. 11C generally illustrates the DNA sensing device of FIG. 8 in a third stage of fabrication.

FIG. 11D generally illustrates the DNA sensing device of FIG. 8 in a fourth stage of fabrication.

FIG. 11E generally illustrates the DNA sensing device of FIG. 8 in a fifth stage of fabrication.

### DETAILED DESCRIPTION

The present disclosure relates generally to a method and apparatus for increasing the lifespan of a nanopore DNA sensing device.

More specific aspects of the disclosure are provided in the following description and related drawings directed to various examples provided for illustration purposes. Alternate aspects may be devised without departing from the scope of the disclosure. Additionally, well-known aspects of the disclosure may not be described in detail or may be omitted so as not to obscure more relevant details.

Those of skill in the art will appreciate that the information and signals described below may be represented using any of a variety of different technologies and techniques. For example, data, instructions, commands, information, signals, bits, symbols, and chips that may be referenced throughout the description below may be represented by voltages, currents, electromagnetic waves, magnetic fields or particles, optical fields or particles, or any combination thereof, depending in part on the particular application, in part on the desired design, in part on the corresponding technology, etc.

Further, many aspects are described in terms of sequences of actions to be performed by, for example, elements of a computing device. It will be recognized that various actions described herein can be performed by specific circuits (e.g., Application Specific Integrated Circuits (ASICs)), by program instructions being executed by one or more processors, or by a combination of both. In addition, for each of the aspects described herein, the corresponding form of any such aspect may be implemented as, for example, “logic configured to” perform the described action.

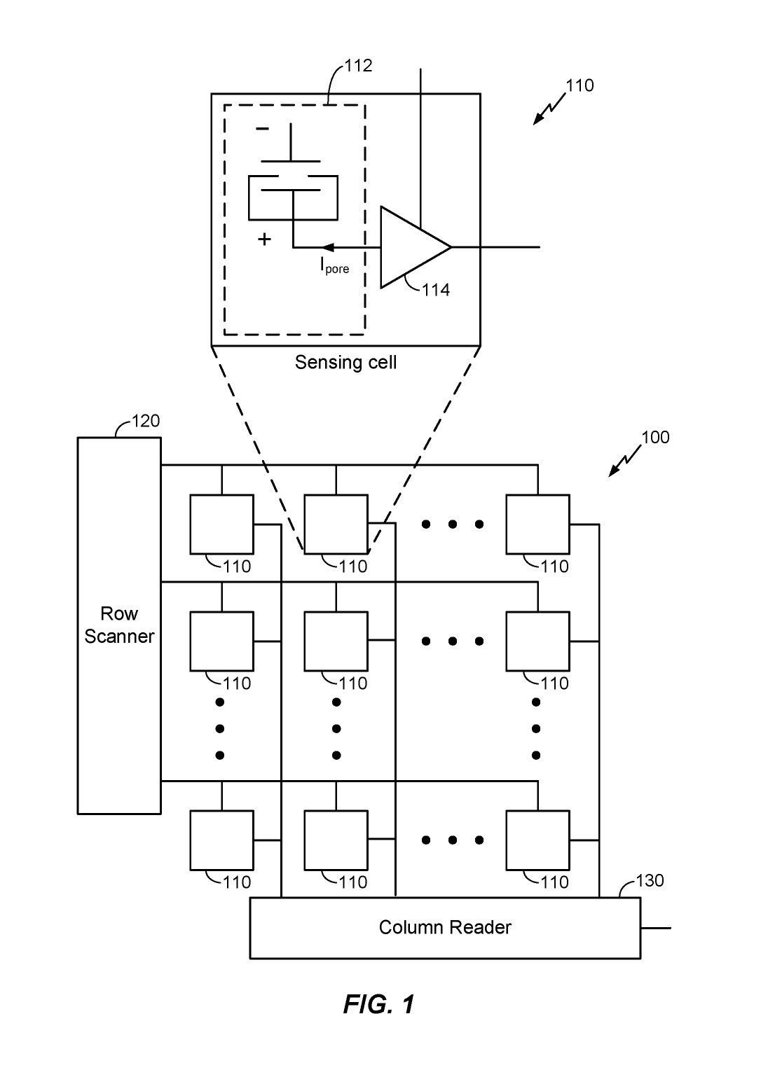

FIG. 1 generally illustrates a DNA sensor array 100 in accordance with aspects of the disclosure.

The DNA sensor array 100 may include a plurality of DNA sensing cells 110. The plurality of DNA sensing cells 110 may be arranged in rows and columns to form a grid pattern. The DNA sensor array 100 may further include a row scanner 120 and a column reader 130. The row scanner 120 and column reader 130 may facilitate the reading of a particular DNA sensing cell 110 from among the plurality of DNA sensing cells 110.

Each DNA sensing cell 110 may include a DNA sensing device 112 and an amplifier 114. As will be described in greater detail below, the DNA sensing device 112 may dynamically generate an electric current as a DNA strand is transmitted through a nanopore in the DNA sensing device 112. Over time, the changes in electric current result in a DNA sensing signal reflecting the particular nucleotides in a particular DNA strand. The amplifier 114 may facilitate amplification of the DNA sensing signal.

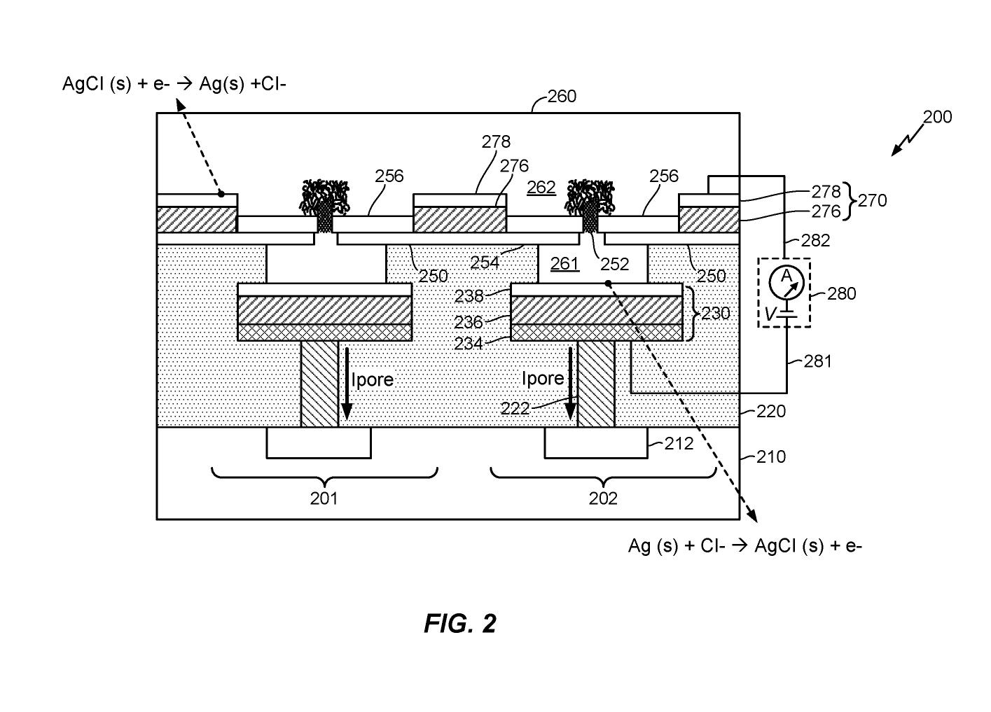

FIG. 2 generally illustrates a pair 200 of nanopore-based DNA sensing devices 201, 202 in accordance with aspects of the disclosure. The DNA sensing devices 201, 202 may be analogous to the DNA sensing device 112 depicted in FIG. 1. The DNA sensing devices 201, 202 may be formed on a substrate 210 and at least partially within an insulator 220. The substrate 210 may include, for example, silicon (Si) and the insulator 220 may include, for example, silicon oxide (SiO₂), silicon mononitride (SiN), a hydrophilic material, any combination thereof, or any other suitable material(s). The DNA sensing devices 201, 202 may be disposed in adjacent DNA sensing cells analogous to the DNA sensing cells 110 depicted in FIG. 1. The DNA sensing devices 201, 202 depicted in FIG. 2 may be substantially similar to one another. Accordingly, the particular components of only one nanopore-based DNA sensing device will be described below.

The DNA sensing device 202 may include a semiconductor device 212 disposed on the substrate 210. The semiconductor device 212 may include, for example, a complementary metal oxide semiconductor (CMOS) transistor. The semiconductor device 212 may be a component of an amplifier analogous to the amplifier 114 depicted in FIG. 1. The insulator 220 may include a via 222 in contact with the semiconductor device 212. The via 222 may include, for example, copper (Cu), tungsten (W), aluminum (Al), any combination thereof, or any other suitable material(s).

The DNA sensing device 202 further includes a first electrode 230 in contact with the via 222. The first electrode 230 may be disposed on or within the insulator 220. The first electrode 230 may include an adhesion/diffusion layer 234, a conductive layer 236, and a surface layer 238.

As an example, the adhesion/diffusion layer 234 may include a chromium (Cr) adhesion layer in contact with the via 222 and a gold (Au) diffusion layer between the conductive layer 236 and the Cr adhesion layer. Additionally or alternatively, the adhesion/diffusion layer 234 may include titanium nitride (TiN) and/or any other suitable material(s). The conductive layer 236 may include silver (Ag), however, it will be understood that any suitable material may be selected. The surface layer 238 may include silver chloride (AgCl), however, it will be understood that any suitable material(s) may be selected.

The DNA sensing device 202 further includes a separation layer 250 having a nanopore 252 embedded therein. The separation layer 250 may include a barrier layer 254 and a hydrophobic layer 256. The barrier layer 254 may include silicon nitride (Si₃N₄), however, it will be understood that any suitable material(s) may be selected. The hydrophobic layer 256 may include a lipid bilayer, a hydrophobic membrane, or any other suitable material(s).

The DNA sensing device 202 further includes a chamber 260. The chamber 260 may hold a conductive fluid therein. The conductive fluid may include, for example, one or more electrolytes, for example, chlorine electrolyte (Cr), potassium electrolyte (K+), hydrogen electrolyte (H+), or any other suitable material. The conductive fluid within the chamber 260 may be divided by the separation layer 250 or a component thereof (for example, the barrier layer 254 and the hydrophobic layer 256) into a first subchamber 261 and a second subchamber 262.

The DNA sensing device 202 may further include a second electrode 270. The second electrode 270 may be disposed on the separation layer 250. The second electrode 270 may include a conductive layer 276 and a surface layer 278. The conductive layer 276 and the surface layer 278 may be analogous to the conductive layer 236 and the surface layer 238 of the first electrode 230. The first electrode 230 may be coupled to a voltage source 280 via a first conductor 281 and the second electrode 270 may be coupled to the voltage source 280 via a second conductor 282.

Fluid in the first subchamber 261 may be in contact with the surface layer 238 of the first electrode 230, and fluid in the second subchamber 262 may be in contact with the surface layer 278 of the second electrode 270. In the DNA sensing device 202 of FIG. 2, the first subchamber 261 may be a positive chamber (i.e., associated with a trans-electrode) and the second subchamber 262 may be a negative chamber (i.e., associated with a cis-electrode), but it will be understood that the polarity of subchambers 261, 262 may be reversed.

Although the chamber 260 is depicted as a closed chamber, it will be understood that this is optional. For example, the chamber 260 may be an open chamber. Moreover, as will be discussed in greater detail below, the chamber 260 may include enough conductive fluid to fill the first subchamber 261 and cover the second electrode 270.

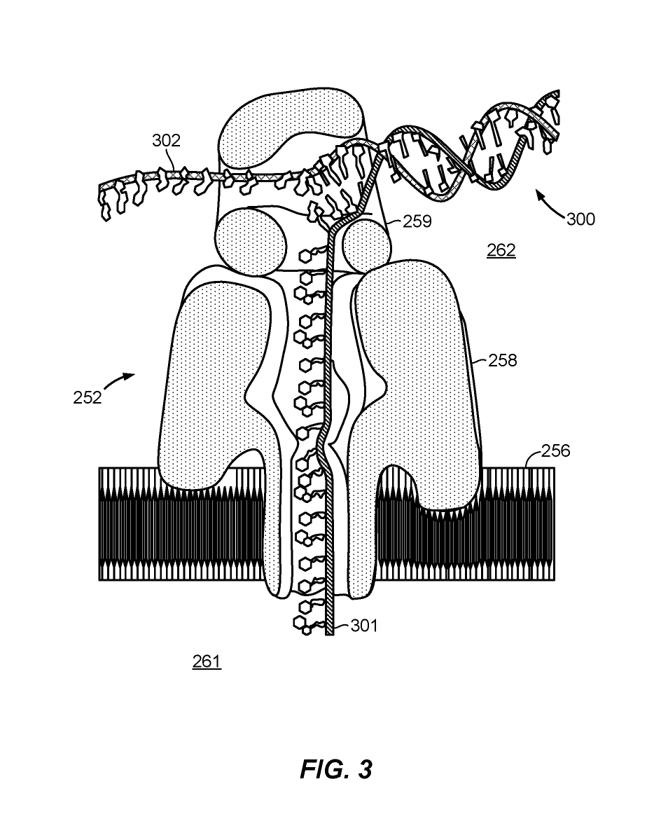

FIG. 3 generally illustrates a detail of the nanopore 252 of FIG. 2 in accordance with an aspect of the disclosure. The nanopore 252 may be a biological nanopore. As noted above, the nanopore 252 may be embedded in the separation layer 250, and the separation layer 250 may separate the first subchamber 261 from the second subchamber 262. As noted previously, the separation layer 250 may include a lipid bilayer, a hydrophobic membrane, or any other suitable material(s).

The nanopore 252 may include, for example, a translocator 258 and an assembler 259. The translocator 258 permits fluid communication (for example, passage of conductive fluid) between the first subchamber 261 and the second subchamber 262. For example, if the second subchamber 262 is negatively charged and the first subchamber 261 is positively charged, then negative ions (for example, Cl⁻) may pass from the second subchamber 262 to the first subchamber 261 via the translocator 258 and/or positive ions (for example, K⁺ and/or H⁺) may pass from the first subchamber 261 to the second subchamber 262. In some implementations, the translocator 258 may include alpha hemolysin.

The assembler 259 may separate a double-stranded DNA molecule 300 into a first DNA strand 301 and a second DNA strand 302 and/or combine the first DNA strand 301 and the second DNA strand 302 into the double-stranded DNA molecule 300. In some implementations, the assembler 259 may include DNA polymerase.

FIG. 3 generally illustrates the separation and/or combination of a double-stranded DNA molecule 300. For example, the double-stranded DNA molecule 300 may move from the second subchamber 262 into the assembler 259, where it is separated by the assembler 259 into the first DNA strand 301 and the second DNA strand 302. The first DNA strand 301 may be led into the translocator 258 and translocated across the separation layer 250, from the second subchamber 262 to the first subchamber 261. As another example, the first DNA strand 301 may be drawn from the first subchamber 261 through the translocator 258 and into the assembler 259, where it is combined with the second DNA strand 302 into the double-stranded DNA molecule 300. The double-stranded DNA molecule 300 may then be moved into the second subchamber 262.

In some implementations, the following method may be used to perform DNA sequencing using the DNA sensing device 202 of FIG. 2 and the nanopore 252 of FIG. 3. First, a voltage may be applied to the first electrode 230 and the second electrode 270 via the first conductor 281 and the second conductor 282, respectively. As a result, a positive charge may appear on the first electrode 230 and a negative charge may appear on the second electrode 270.

As an example, the second electrode 270 may include a surface layer 278 including AgCl and a conductive layer 276 including Ag. When the voltage V is applied (such that the second electrode 270 is negatively charged), the AgCl in the second electrode 270 may be converted into Ag and chlorine electrolytes, i.e., AgCl(s)+e⁻→Ag(s)+Cl⁻. As the second electrode 270 generates Cl⁻ ions, the second subchamber 262 may become negatively charged.

Moreover, the first electrode 230 may include a surface layer 238 including AgCl and a conductive layer 236 including Ag. When the voltage V is applied (such that the first electrode 230 is positively charged), the Ag in the first electrode 230 may combine with Cl⁻ ions in the first subchamber 261, i.e., Ag(s)+Cl⁺→AgCl(s)+e⁻. As the first electrode 230 combines Cl⁻ ions into AgCl, the first subchamber 261 may become positively charged.

As a result, ions in the chamber 260 may have a tendency to flow toward either the first subchamber 261 (which is positively charged) or the second subchamber 262 (which is negatively charged). For example, Cl⁻ ions in the chamber 260 (including Cl⁻ ions generated at the second electrode 270) may have a tendency to flow toward the positively-charged first subchamber 261.

As the first electrode 230 generates electrons e⁻, an electrical current i_{PORE} may flow through the via 222 to the semiconductor device 212.

Because Cl⁻ ions may have a tendency to flow toward the positively-charged first subchamber 261, the Cl⁻ ions may translocate across the separation layer 250 via the nanopore 252. However, the nanopore 252 may also be configured to translocate DNA (for example, the first DNA strand 301, as shown in FIG. 3).

As the first DNA strand 301 shown in FIG. 3 is being translocated, it may impede the flow of Cl⁻ ions through the nanopore 252. As a result, the current i_{PORE} may be reduced due to the translocation of the first DNA strand 301. Moreover, different types of nucleotide may have different effects on the flow of Cl⁻ ions through the nanopore 252.

Accordingly, as different types of nucleotide pass through the nanopore 252, different quantities of Cl⁻ ions may pass through the nanopore 252, and a different electrical current i_{PORE} may be measured at the semiconductor device 212. For example, a C nucleotide may cause a current i_{C}, an A nucleotide may cause a current i_{A}, a T nucleotide may cause a current i_{T}, and a G nucleotide may cause a small current i_{G}. As the first DNA strand 301 passes through the nanopore 252, the DNA sensing device 202 will generate a current waveform i_{PORE}(t) that indicates the sequence of nucleotides in the first DNA strand 301.

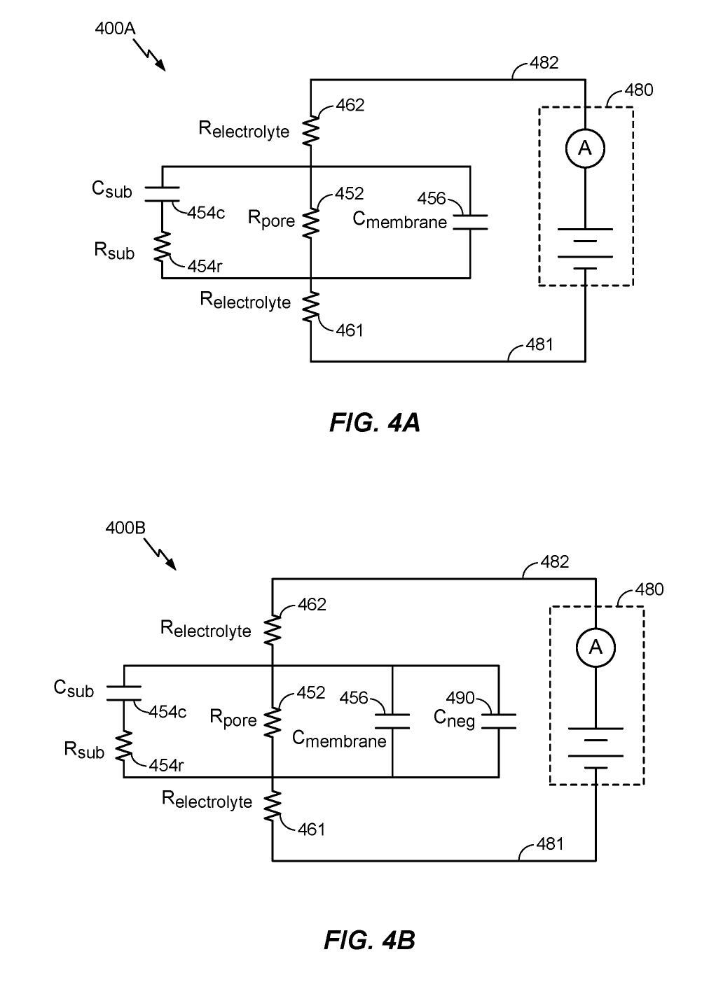

FIG. 4A generally illustrates a circuitry model 400A associated with the nanopore-based DNA sensing device 202 of FIG. 2.

The circuitry model 400 may include a voltage source 480, a first conductor 481, and a second conductor 482. The voltage source 480, the first conductor 481, and the second conductor 482 may be analogous to the voltage source 280, the first conductor 281, and the second conductor 282 depicted in FIG. 2. As shown in FIG. 2, the first conductor 281 and the second conductor 282 may be coupled to the first electrode 230 and the second electrode 270, respectively, thereby causing a flow of ions from the first subchamber 261 to the second subchamber 262, or vice-versa.

The flow of ions between the first subchamber 261 to the second subchamber 262 may be affected by resistances and/or capacitances associated with various components of the DNA sensing device 202 depicted in FIG. 2. For example, a first electrolyte resistance 461 and a second electrolyte resistance 462 may represent a resistance to ion flow associated with the first subchamber 261 and the second subchamber 262, respectively. A barrier layer capacitance 454c and a barrier layer resistance 454r may represent a capacitance of and resistance to ion flow associated with the barrier layer 254, one or more components thereof, other elements of the DNA sensing device 202, or any combination thereof. A pore resistance 452 may represent a resistance to ion flow associated with the nanopore 252. A membrane capacitance 456 may represent a capacitance of ion flow associated with the hydrophobic layer 256.

The resistances and capacitances depicted in the circuitry model 400A of FIG. 4A may limit the bandwidth of a DNA sensing signal associated with the DNA sensing device 202 by reducing the maximum cutoff frequency of the DNA sensing signal and increasing ionic current noise within the DNA sensing signal.

For example, the maximum cutoff frequency may be inversely proportional to 2πRC, where R is the total resistance associated with the circuitry model 400 and C is the total capacitance of the circuitry model 400. As an example, the first electrolyte resistance 461 and the second electrolyte resistance 462 may be in the range of 0.1-1.0 kΩ, the pore resistance 452 may be equal to or on the order of 1.0 GΩ, and the membrane capacitance 456 may be in the range of 18-30 fF. The effects associated with the barrier layer capacitance 454c and the barrier layer resistance 454r may be negligible by comparison to the effects of the aforementioned resistances and capacitances, resulting in a cutoff frequency equal to or on the order of 5.5 KHz.

In some implementations, the optimal cutoff frequency may be equal or nearer to the cutoff frequency associated with the supporting electronics associated with, for example, the DNA sensor array 100 and/or the DNA sensing device 202. For example, in some implementations, the cutoff frequency associated with the DNA sensor array 100 and/or the DNA sensing device 202 may be equal to or on the order of 100 KHz. In such an implementation, it may be advantageous to increase the cutoff frequency associated with the circuitry model 400.

FIG. 4B generally illustrates a circuitry model 400B with negative capacitance in accordance with aspects of the disclosure.

As noted above with respect to FIG. 4A, the DNA sensing device 202 depicted in FIG. 2 may be associated with resistances and capacitances that decrease the maximum cutoff frequency and increase the noise associated with the DNA sensing device 202. Accordingly, new technologies are needed for improving the DNA sensing signal of nanopore-based DNA sensing devices.

In accordance with aspects of the disclosure, a negative capacitance 490 may be added to the circuitry model 400A (as depicted in FIG. 4A) to provide the circuitry model 400B (as depicted in FIG. 4B). The negative capacitance 490 may offset the positive capacitances associated with the barrier layer capacitance 454c, the membrane capacitance 456, or any combination thereof. For example, if the combined capacitance of the membrane capacitance 456 and the barrier layer capacitance 454c is equal to 30 fF, then a negative capacitance 490 of −30 fF may be added in parallel with the membrane capacitance 456 and the barrier layer capacitance 454c. The effect of the negative capacitance 490 may be to increase the maximum cutoff frequency and/or decrease noise.

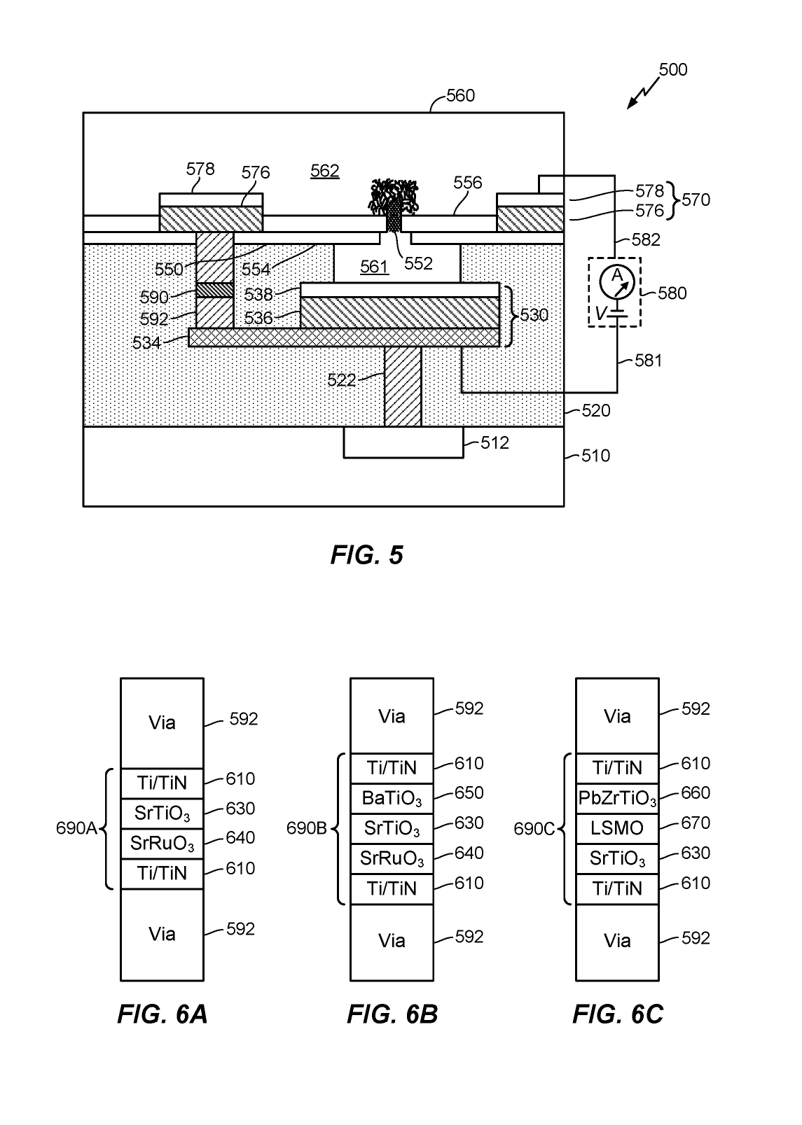

FIG. 5 generally illustrates a DNA sensing device 500 with a negative capacitance layer 590 in accordance with aspects of the disclosure. The DNA sensing device 500 may have a number of components that are analogous to the components of the DNA sensing device 202. For example, the DNA sensing device 500 may include a substrate 510, a semiconductor device 512, an insulator 520, a via 522, a first electrode 530, an adhesion/diffusion layer 534, a conductive layer 536, a surface layer 538, a separation layer 550, a nanopore 552, a barrier layer 554, a hydrophobic layer 556, a chamber 560, a first subchamber 561, a second subchamber 562, a second electrode 570, a conductive layer 576, a surface layer 578, a voltage source 580, a first conductor 581, and a second conductor 582. These elements depicted in FIG. 5 may be analogous in some respects to the substrate 210, the semiconductor device 212, the insulator 220, the via 222, the first electrode 230, the adhesion/diffusion layer 234, the conductive layer 236, the surface layer 238, the separation layer 250, the nanopore 252, the barrier layer 254, the hydrophobic layer 256, the chamber 260, the first subchamber 261, the second subchamber 262, the second electrode 270, the conductive layer 276, the surface layer 278, the voltage source 280, the first conductor 281, and the second conductor 282 depicted in FIG. 2. For brevity, only the differences will be described.

As noted above with respect to FIG. 4B, the negative capacitance 490 may be added to the circuitry model 401 in parallel with the membrane capacitance 456 and the barrier layer capacitance 454c. The effect of the negative capacitance 490 may be to increase the maximum cutoff frequency and/or decrease noise. Accordingly, FIG. 5 depicts a negative capacitance layer 590 disposed between the first electrode 530 and the second electrode 570. The negative capacitance layer 590 may constitute means for causing a negative capacitance. For example, the DNA sensing device 500 may include a via 592 (analogous to the via 522) that couples at least a portion of the first electrode 530 to a first terminal of the negative capacitance layer 590 and at least a portion of the second electrode 570 to a second terminal of the negative capacitance layer 590. The via 592 may traverse at least a portion of the insulator 520, at least a portion of the separation layer 550, at least a portion of the barrier layer 554, or any combination thereof. For example, as depicted in FIG. 5, the via 592 may couple the negative capacitance layer 590 to the adhesion/diffusion layer 534 of the first electrode 530 and the conductive layer 576 of the second electrode 570.

As will be understood from FIG. 5, by disposing the negative capacitance layer 590 between the first electrode 530 and the second electrode 570, the capacitance associated with the DNA sensing device 500 may be offset, thereby increasing the maximum cutoff frequency of the DNA sensing device 500 and/or decreasing noise associated with the DNA sensing device 500. The negative capacitance layer 590 may include any suitable material(s). For example, the negative capacitance layer 590 may include a strontium titanate layer, a strontium ruthenate layer, a barium titanate layer, a lead zirconate titanate layer, a lanthanum strontium manganite layer, or any combination thereof.

FIGS. 6A-6C generally illustrate three alternative arrangements for implementing a negative capacitance layer analogous to the negative capacitance layer 590 depicted in FIG. 5. In each arrangement, the negative capacitance layer is disposed within the via 592 depicted in FIG. 5.

FIG. 6A generally illustrates a negative capacitance layer 690A in accordance with aspects of the disclosure. The negative capacitance layer 690A may constitute means for causing a negative capacitance. The negative capacitance layer 690A may be disposed within the via 592 depicted in FIG. 5 and may include a plurality of sublayers, as will be described in greater detail below. Opposing surfaces of the negative capacitance layer 690A may be coupled to the via 592 through a pair of adhesion/diffusion barriers 610. The opposing surfaces may constitute a first terminal and a second terminal of the negative capacitance layer 690A. The adhesion/diffusion barriers 610 may be constructed of any suitable material, for example, titanium (Ti), titanium nitride (TiN), or any combination thereof.

Between the pair of adhesion/diffusion barriers 610, the negative capacitance layer 690A may include a strontium titanate layer 630 (for example, chemical compound SrTiO₃) coupled to a strontium ruthenate layer 640 (for example, chemical compound SrRuO₃). The strontium titanate layer 630 may be coupled to a first adhesion/diffusion barrier of the pair of adhesion/diffusion barriers 610 and the strontium ruthenate layer 640 may be coupled to a second adhesion/diffusion barrier of the pair of adhesion/diffusion barriers 610.

FIG. 6B generally illustrates a negative capacitance layer 690B in accordance with aspects of the disclosure. The negative capacitance layer 690B may constitute means for causing a negative capacitance. The negative capacitance layer 690B may be disposed within the via 592 depicted in FIG. 5 and may include a plurality of sublayers, as will be described in greater detail below. Opposing surfaces of the negative capacitance layer 690B may be coupled to the via 592 through the pair of adhesion/diffusion barriers 610 described above. The opposing surfaces may constitute a first terminal and a second terminal of the negative capacitance layer 690B.

Like the negative capacitance layer 690A, the negative capacitance layer 690B may include a strontium titanate layer 630 coupled to a strontium ruthenate layer 640. Moreover, the strontium ruthenate layer 640 may be coupled to a second adhesion/diffusion barrier of the pair of adhesion/diffusion barriers 610. However, unlike the negative capacitance layer 690A, the negative capacitance layer 690B may include a barium titanate layer 650 (for example, chemical compound BaTiO₃) between the strontium titanate layer 630 and the first adhesion/diffusion barrier of the pair of adhesion/diffusion barriers 610.

FIG. 6C generally illustrates a negative capacitance layer 690C in accordance with aspects of the disclosure. The negative capacitance layer 690C may constitute means for causing a negative capacitance. The negative capacitance layer 690C may be disposed within the via 592 depicted in FIG. 5 and may include a plurality of sublayers, as will be described in greater detail below. Opposing surfaces of the negative capacitance layer 690C may be coupled to the via 592 through the pair of adhesion/diffusion barriers 610 described above. The opposing surfaces may constitute a first terminal and a second terminal of the negative capacitance layer 690C.

Like the negative capacitance layer 690A, the negative capacitance layer 690C may include a strontium titanate layer 630. The negative capacitance layer 690C may further include a lead zirconate titanate layer 660 (for example, chemical compound PbZrTiO₃) and a lanthanum strontium manganite layer 670 (for example, chemical compound LSMO). The lead zirconate titanate layer 660 may be coupled to a first adhesion/diffusion barrier of the pair of adhesion/diffusion barriers 610 and the strontium titanate layer 630 may be coupled to a second adhesion/diffusion barrier of the pair of adhesion/diffusion barriers 610. The lanthanum strontium manganite layer 670 may be disposed between and in contact with the lead zirconate titanate layer 660 and the strontium titanate layer 630.

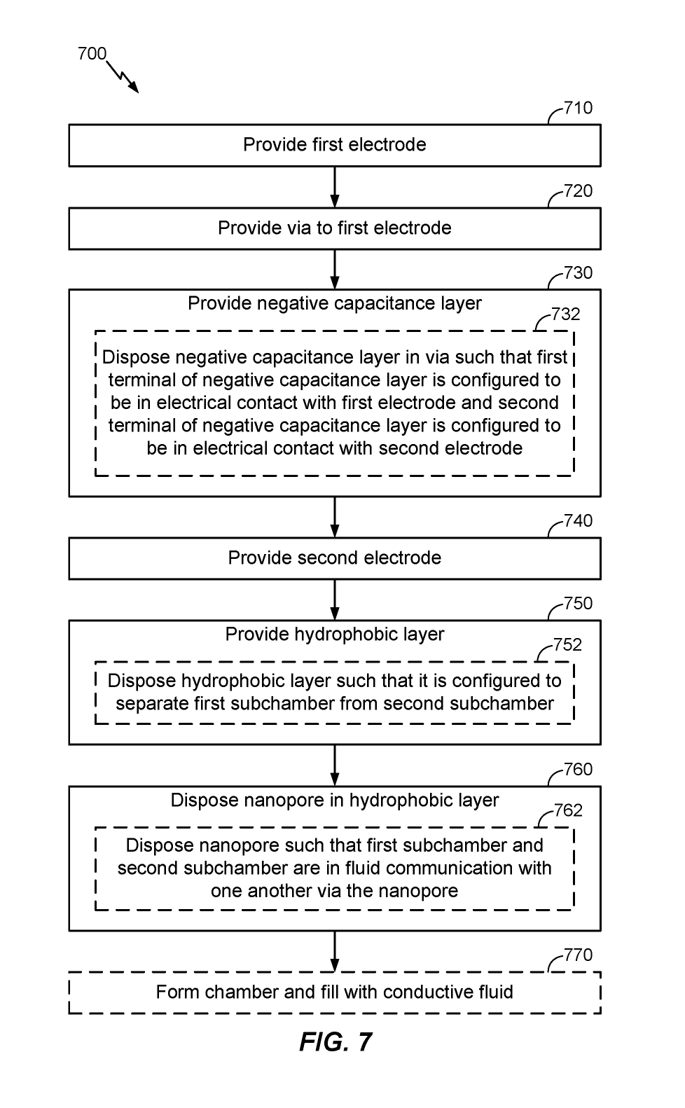

FIG. 7 generally illustrates a method 700 for fabricating the DNA sensing device of FIG. 5.

At 710, the method 700 provides the first electrode 530. As will be understood from FIG. 5, the first electrode 530 may be disposed on or within the insulator 520. Moreover, the first electrode 530 or a portion thereof (for example, the adhesion/diffusion layer 534) may be electrically coupled to the semiconductor device 512 through the via 522. As depicted in FIG. 5, a portion of the first electrode 530 may be disposed beneath a cavity within the insulator 520 which is to form the first subchamber 561. Moreover, another portion of the first electrode 530 (for example, the adhesion/diffusion layer 534) may be disposed such that it is not beneath the cavity.

At 720, the method 700 provides the via 592 to the first electrode 530. As depicted in FIG. 5, the via 592 may be disposed through the insulator 520, the separation layer 550, or any combination thereof. Moreover, the via 592 may disposed such that it extends to the adhesion/diffusion layer 534 of the first electrode 530.

At 730, the method 700 provides the negative capacitance layer 590.

At 732, the method 700 optionally disposes the negative capacitance layer 590 in the via 592 such that a first terminal of negative capacitance layer 590 is configured to be in electrical contact with the first electrode 530 and a second terminal of the negative capacitance layer 590 is configured to be in electrical contact with the second electrode 570. For example, the via 592 may be filled with conductive material including a first portion of conductive material configured to electrically couple a bottom surface of the negative capacitance layer 590 with the first electrode 530 and a second portion of conductive material configured to electrically couple a top surface of the negative capacitance layer 590 with the second electrode 570.

At 740, the method 700 provides the second electrode 570. The second electrode 570 may be disposed on the separation layer 550 or a portion thereof (for example, the barrier layer 554. The barrier layer 554 may itself be disposed, at least in part, on the insulator 520. At least a portion of the second electrode 570 may be disposed above at least a portion of the first electrode 530. Moreover, at least a portion of the second electrode 570 may be disposed above the via 592. As noted above, the via 592 may be disposed through the separation layer 550 and the insulator 520. Accordingly, the second electrode 570 may be in electrical contact with the negative capacitance layer 590 through the via 592.

At 750, the method 700 provides the hydrophobic layer 556. At least a portion of the hydrophobic layer 556 may be disposed on at least a portion of the barrier layer 554.

At 752, the method 700 optionally disposes the hydrophobic layer 556 such that it is configured to separate the first subchamber 561 from the second subchamber 562. As depicted in FIG. 5, the hydrophobic layer 556 may be disposed above a cavity in the insulator 520 that includes the first electrode 530, thereby defining the first subchamber 561.

At 760, the method 700 disposes the nanopore 552 in the hydrophobic layer 556. As depicted in FIG. 5, the nanopore 552 may be disposed in a portion of the hydrophobic layer 556 that is not disposed on the barrier layer 554. Moreover, the nanopore 552 may be disposed above a cavity in the insulator 520 that includes the first electrode 530.

At 762, the method 700 optionally disposes the nanopore 552 such that the first subchamber 561 and the second subchamber 562 are in fluid communication with one another via the nanopore 552. A depicted in FIG. 5, the nanopore 552 may be disposed such that fluid in the first subchamber 561 can move into or out of the first subchamber 561 through the nanopore 552.

At 770, the method 700 optionally forms the chamber 560 and fills the chamber 560 with conductive fluid.

Although the method 700 is depicted in FIG. 7 as if it is to be performed in a specific order, it will be understood that the method 700 may be performed in any suitable sequence, including sequences other than the sequence depicted in FIG. 7. For example, the optional formation of the chamber 560 at 770 may be performed prior to the disposing of the hydrophobic layer 556 at 750.

FIG. 8 generally illustrates another DNA sensing device 800 with a negative capacitance layer 890 in accordance with aspects of the disclosure. The negative capacitance layer 890 may constitute means for causing a negative capacitance. The DNA sensing device 800 may have a number of components that are analogous to the components of the DNA sensing device 202. For example, the DNA sensing device 800 may include an insulator 820, a nanopore 852, a hydrophobic layer 856, a first subchamber 861, and a second subchamber 862. These elements depicted in FIG. 8 may be analogous in some respects to the insulator 220, the nanopore 252, the hydrophobic layer 256, the first subchamber 261, and the second subchamber 262 depicted in FIG. 2. The DNA sensing device 800 may be disposed in relation to other elements analogous to the elements depicted in FIG. 2, such as the substrate 210, the semiconductor device 212, the via 222, the first electrode 230, the adhesion/diffusion layer 234, the conductive layer 236, the surface layer 238, the separation layer 250, the chamber 260, the second electrode 270, the conductive layer 276, the surface layer 278, the voltage source 280, the first conductor 281, and the second conductor 282. For brevity, only the differences will be described. However, the barrier layer 254 may be omitted from the DNA sensing device 800 and a negative capacitance layer 890 may be substituted for the barrier layer 254.

As noted above with respect to FIG. 4B, the negative capacitance 490 may be added to the circuitry model 401 in parallel with the membrane capacitance 456 and the barrier layer capacitance 454c. It will be understood that the membrane capacitance 456 and the negative capacitance 490 are circuitry models and are depicted in FIG. 8 solely for illustrative purposes.

FIG. 8 depicts the negative capacitance layer 890 disposed between the first subchamber 861 (which may be in contact with an electrode analogous to the first electrode 230) and the second subchamber 862 (which may be in contact with an electrode analogous to the second electrode 270). A portion of the negative capacitance layer 890 in contact with the first subchamber 861 may constitute a first terminal of the negative capacitance layer 890 and a portion of the negative capacitance layer 890 in contact with the second subchamber 862 may constitute a second terminal of the negative capacitance layer 890. Accordingly, the negative capacitance layer 890 may be disposed in parallel with, for example, the membrane capacitance 456 associated with the hydrophobic layer 856, and the effect of the negative capacitance layer 890 may be to increase the maximum cutoff frequency of the DNA sensing device 800 and/or decrease noise in the DNA sensing device 800.

The negative capacitance layer 890 may include any suitable material(s). For example, the negative capacitance layer 890 may include a strontium titanate layer, a strontium ruthenate layer, a barium titanate layer, a lead zirconate titanate layer, a lanthanum strontium manganite layer, or any combination thereof.

The negative capacitance layer 890 may include opposing surfaces, and at least a portion of each of the opposing surfaces may form a first terminal or a second terminal of the negative capacitance layer 890. For example, a bottom surface of the negative capacitance layer 890 may be disposed on the insulator 820 and the portion of the bottom surface that is not disposed on the insulator 820 may be in contact with the first subchamber 861. Moreover, the hydrophobic layer 856 may be disposed on at least a portion of the negative capacitance layer 890. The portion of the negative capacitance layer 890 that is not in contact with the hydrophobic layer 856 may be in contact with the second subchamber 862. As will be further understood from FIG. 8, the portions of the negative capacitance layer 890 that are in contact with the first subchamber 861 on one surface and in contact with the second subchamber 862 on the opposing surface will act as a negative capacitor analogous to the negative capacitance 490 depicted in FIG. 4B.

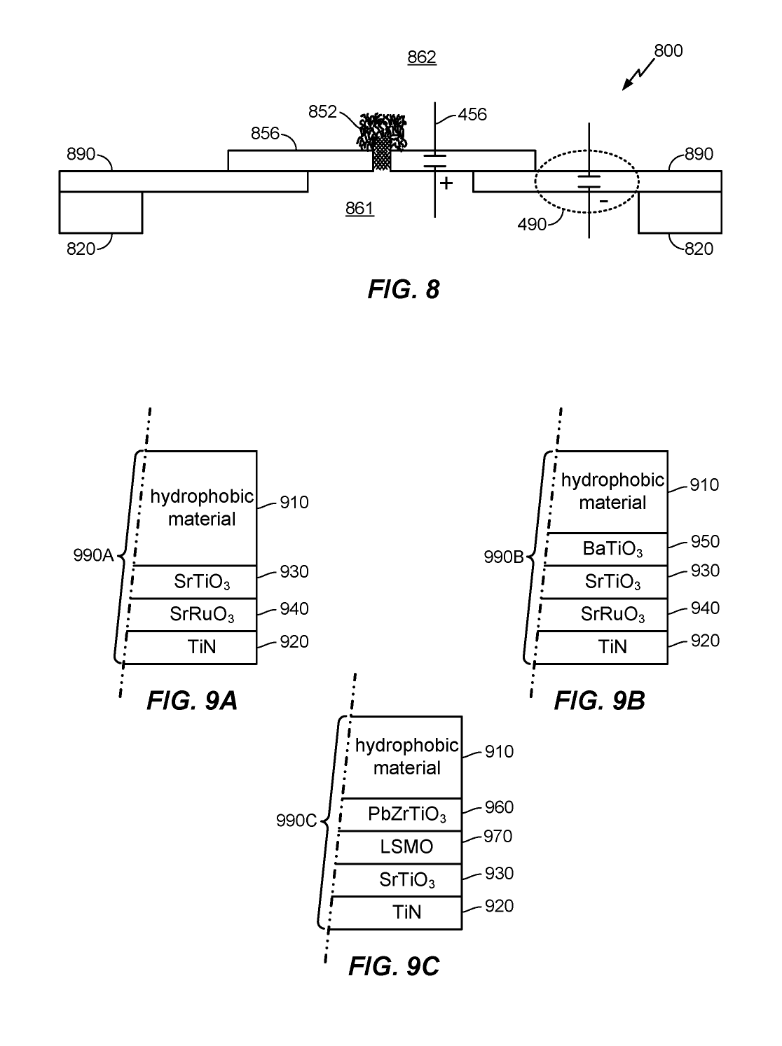

FIGS. 9A-9C generally illustrate three alternative arrangements for implementing a negative capacitance layer analogous to the negative capacitance layer 890 depicted in FIG. 8. In each arrangement, the negative capacitance layer is disposed between the first subchamber 861 (which may be in contact with an electrode analogous to the first electrode 230) and the second subchamber 862 (which may be in contact with an electrode analogous to the second electrode 270), as depicted in FIG. 8.

FIG. 9A generally illustrates a negative capacitance layer 990A in accordance with aspects of the disclosure. The negative capacitance layer 990A may constitute means for causing a negative capacitance. The negative capacitance layer 990A may include a hydrophobic material layer 910. The hydrophobic material layer 910 may form a surface (for example, a first surface) of the negative capacitance layer 990A and may be in contact with the first subchamber 861 or the second subchamber 862. An opposing surface (for example, a second surface) of the negative capacitance layer 990A may include an adhesion/diffusion barrier 920. The adhesion/diffusion barrier 920 may be constructed of any suitable material, for example, titanium (Ti), titanium nitride (TiN), or any combination thereof.

Between the hydrophobic material layer 910 and the adhesion/diffusion barrier 920, the negative capacitance layer 990A may include a strontium titanate layer 930 (for example, chemical compound SrTiO₃) coupled to a strontium ruthenate layer 940 (for example, chemical compound SrRuO₃). The strontium titanate layer 930 may be coupled to the hydrophobic material layer 910 and the strontium ruthenate layer 940 may be coupled to the adhesion/diffusion barrier 920.

FIG. 9B generally illustrates a negative capacitance layer 990B in accordance with aspects of the disclosure. The negative capacitance layer 990B may constitute means for causing a negative capacitance. The negative capacitance layer 990B may include the hydrophobic material layer 910 described above in relation to FIG. 9A. The hydrophobic material layer 910 may be a surface of the negative capacitance layer 990B and may be in contact with the first subchamber 861 or the second subchamber 862. An opposing surface of the negative capacitance layer 990B may include the adhesion/diffusion barrier 920 described above in relation to FIG. 9A. The opposing surfaces may constitute a first terminal and a second terminal of the negative capacitance layer 990B.

Between the hydrophobic material layer 910 and the adhesion/diffusion barrier 920, the negative capacitance layer 990A may include the strontium titanate layer 930 and the strontium ruthenate layer 940 described above in relation to FIG. 9A. However, unlike the negative capacitance layer 990A, the negative capacitance layer 990B may include a barium titanate layer 950 between the strontium titanate layer 930 and the hydrophobic material layer 910.

FIG. 9C generally illustrates a negative capacitance layer 990C in accordance with aspects of the disclosure. The negative capacitance layer 990C may constitute means for causing a negative capacitance. The negative capacitance layer 990C may include the hydrophobic material layer 910 described above in relation to FIG. 9A. The hydrophobic material layer 910 may be a surface of the negative capacitance layer 990C and may be in contact with the first subchamber 861 or the second subchamber 862. An opposing surface of the negative capacitance layer 990C may include the adhesion/diffusion barrier 920 described above in relation to FIG. 9A. The opposing surfaces may constitute a first terminal and a second terminal of the negative capacitance layer 990C.

Between the hydrophobic material layer 910 and the adhesion/diffusion barrier 920, the negative capacitance layer 990C may include the strontium titanate layer 930 described above in relation to FIG. 9A. However, unlike the negative capacitance layer 990A, the negative capacitance layer 990C may further include a lead zirconate titanate layer 960 and a lanthanum strontium manganite layer 970. The lead zirconate titanate layer 960 may be coupled to the hydrophobic material layer 910 and the strontium titanate layer 930 may be coupled to the adhesion/diffusion barrier 920. The lanthanum strontium manganite layer 970 may be disposed between and in contact with the lead zirconate titanate layer 960 and the strontium titanate layer 930.

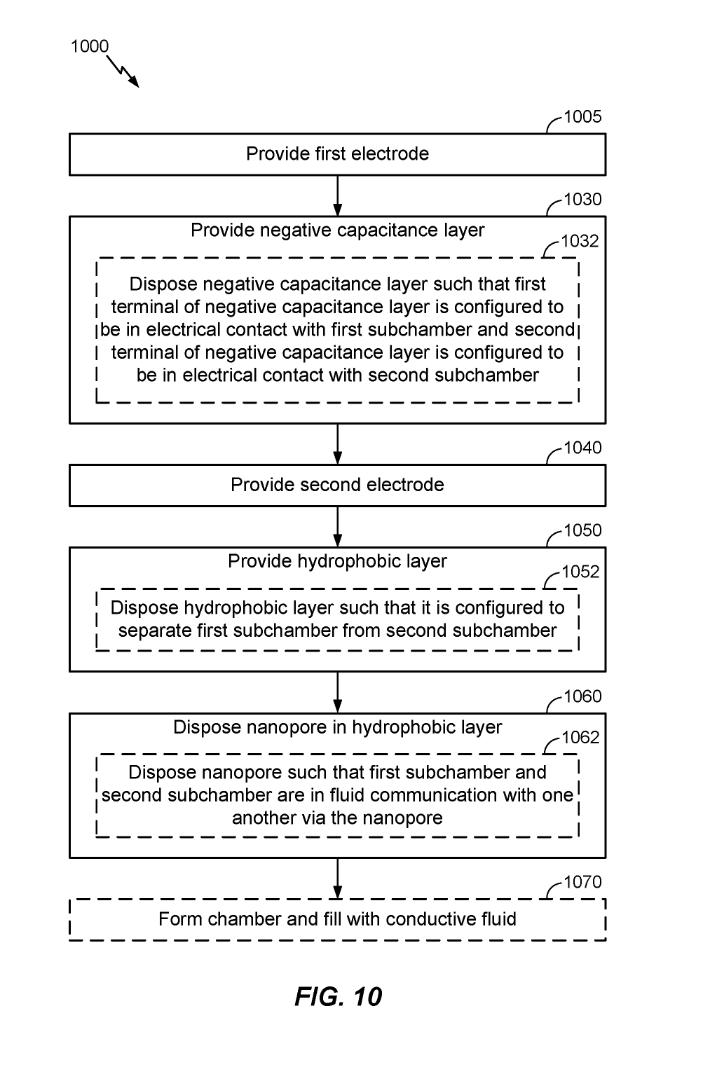

FIG. 10 generally illustrates a method for fabricating the DNA sensing device of FIG. 8.

At 1010, the method 1000 provides a first electrode analogous to the first electrode 230. As will be understood from the previous discussion, the first electrode may be disposed on or within the insulator 820. Moreover, the first electrode or a portion thereof (for example, an adhesion/diffusion layer analogous to adhesion/diffusion layer 234) may be electrically coupled to a semiconductor device analogous to the semiconductor device 212 through a via analogous to the via 222. A portion of the first electrode may be disposed beneath a cavity within the insulator 820 which is to form the first subchamber 861.

At 1030, the method 1000 provides the negative capacitance layer 890. As depicted in FIG. 8, the negative capacitance layer 890 may be disposed on, for example, the insulator 820.

At 1032, the method 1000 optionally disposes the negative capacitance layer 890 such that a first terminal of the negative capacitance layer 890 is configured to be in electrical contact with the first subchamber 861 and a second terminal of the negative capacitance layer 890 is configured to be in electrical contact with the second subchamber 862. For example, the negative capacitance layer 890 may be disposed on the insulator 820.

At 1040, the method 1000 provides a second electrode analogous to the second electrode 270. The second electrode may be disposed on the negative capacitance layer 890 or a portion thereof.

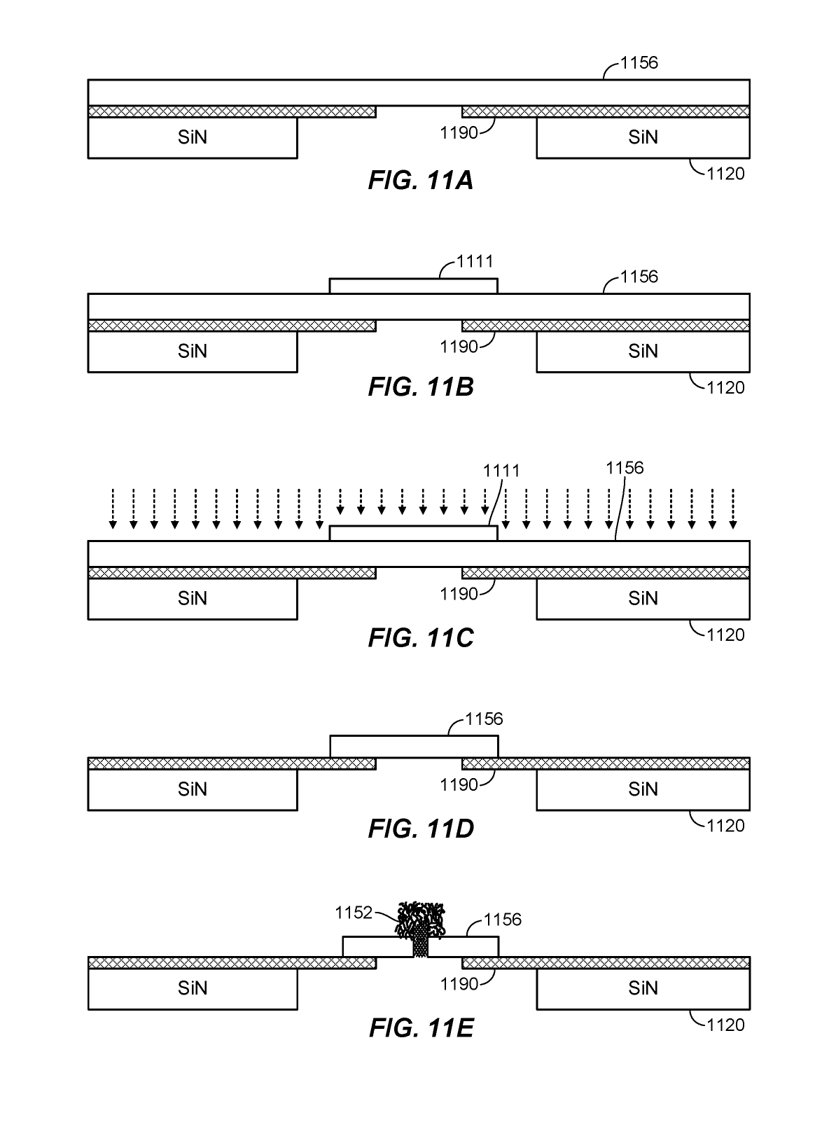

At 1050, the method 1000 provides the hydrophobic layer 856. At least a portion of the hydrophobic layer 856 may be disposed on at least a portion of the negative capacitance layer 890. The hydrophobic layer 856 may be deposited and shaped as will be discussed in greater below with respect to FIGS. 11A-11E.

At 1052, the method 1000 optionally disposes the hydrophobic layer 856 such that it is configured to separate the first subchamber 861 from the second subchamber 862. As depicted in FIG. 8, the hydrophobic layer 856 may be disposed above a cavity in the insulator 820 that includes the first electrode, thereby defining the first subchamber 861.

At 1060, the method 1000 disposes the nanopore 852 in the hydrophobic layer 856. As depicted in FIG. 8, the nanopore 852 may be disposed in a portion of the hydrophobic layer 856 that is not disposed on the negative capacitance layer 890. Moreover, the nanopore 852 may be disposed above a cavity in the insulator 820 that includes the first electrode.

At 1062, the method 1000 optionally disposes the nanopore 852 such that the first subchamber 861 and the second subchamber 862 are in fluid communication with one another via the nanopore 852. A depicted in FIG. 8, the nanopore 852 may be disposed such that fluid in the first subchamber 861 can move into or out of the first subchamber 861 through the nanopore 852.

At 1070, the method 1000 optionally forms a chamber analogous to the chamber 260 and fills the chamber with conductive fluid.

Although the method 1000 is depicted in FIG. 10 as if it is to be performed in a specific order, it will be understood that the method 1000 may be performed in any suitable sequence, including sequences other than the sequence depicted in FIG. 10. For example, the optional formation of the chamber at 1070 may be performed prior to the providing of the hydrophobic layer 856 at 1052.

FIGS. 11A-11E generally illustrate the DNA sensing device 800 of FIG. 8 in various stages of fabrication, as will be described in greater detail below.

FIG. 11A generally illustrates the DNA sensing device 800 of FIG. 8 in a first stage of fabrication. In the first stage, an insulator 1120, negative capacitance layer 1190, and hydrophobic layer 1156 may be provided. The insulator 1120 and hydrophobic layer 1156 may be analogous to the insulator 820 and hydrophobic layer 856 depicted in FIG. 8. The negative capacitance layer 1190 may be analogous to the negative capacitance layer 890 depicted in FIG. 8 and/or any of the negative capacitance layers 990A, 990B, 990C depicted in FIGS. 9A-9C.

FIG. 11B generally illustrates the DNA sensing device 800 of FIG. 8 in a second stage of fabrication. In the second stage a mask 1111 may be provided. The mask 1111 may be photoresistant to one or more frequencies of light to which the hydrophobic layer 1156 is not photoresistant. The size of the mask 1111 may be selected such that the negative capacitance layer 1190 has an optimal capacitance value. For example, by increasing the size of the mask 1111, the effective area of the negative capacitance layer 1190 may be decreased, and by decreasing the size of the mask 1111, the effective area of the negative capacitance layer 1190 may be increased.

FIG. 11C generally illustrates the DNA sensing device 800 of FIG. 8 in a third stage of fabrication. In the third stage, the mask 1111 and at least a portion of the hydrophobic layer 1156 not covered by the mask 1111 may be exposed to light. The light may include one or more frequencies to which the mask 1111 is photoresistant and to which the 1156 is not photoresistant.

FIG. 11D generally illustrates the DNA sensing device 800 of FIG. 8 in a fourth stage of fabrication. In the fourth stage, the portion of the hydrophobic layer 1156 not covered by the mask 1111 and the mask 1111 itself have been removed.

FIG. 11E generally illustrates the DNA sensing device 800 of FIG. 8 in a fifth stage of fabrication. In the fifth stage a nanopore 1152 is provided in the hydrophobic layer 1156. The nanopore 1152 may be analogous to the nanopore 852 depicted in FIG. 8.

While the foregoing disclosure shows various illustrative aspects, it should be noted that various changes and modifications may be made to the illustrated examples without departing from the scope defined by the appended claims. The present disclosure is not intended to be limited to the specifically illustrated examples alone. For example, unless otherwise noted, the functions, steps, and/or actions of the method claims in accordance with the aspects of the disclosure described herein need not be performed in any particular order. Furthermore, although certain aspects may be described or claimed in the singular, the plural is contemplated unless limitation to the singular is explicitly stated.

What is claimed is:

1. A DNA sensing device, comprising:
a first electrode;
a second electrode;
a hydrophobic layer having a nanopore disposed therein; and
a negative capacitance layer.

2. The DNA sensing device of claim 1, further comprising a chamber divided into a first subchamber and a second subchamber.

3. The DNA sensing device of claim 2, wherein the first subchamber and the second subchamber are separated at least in part by the hydrophobic layer.

4. The DNA sensing device of claim 2, wherein the first subchamber and the second subchamber are in fluid communication with one another via the nanopore.

5. The DNA sensing device of claim 2, wherein the first electrode is in contact with the first subchamber and the second electrode is in contact with the second subchamber.

6. The DNA sensing device of claim 5, further comprising a voltage source electrically coupled to the first electrode and the second electrode.

7. The DNA sensing device of claim 1, wherein a first terminal of the negative capacitance layer is in electrical contact with the first electrode and a second terminal of the negative capacitance layer is in electrical contact with the second electrode.

8. The DNA sensing device of claim 7, further comprising a via configured to electrically couple the first terminal of the negative capacitance layer to the first electrode and the second terminal of the negative capacitance layer to the second electrode, wherein the negative capacitance layer is disposed in the via.

9. The DNA sensing device of claim 7, wherein the first terminal includes a first adhesion/diffusion layer and the second terminal includes a second adhesion/diffusion layer, the negative capacitance layer further comprising a strontium titanate layer, a strontium ruthenate layer, a barium titanate layer, a lead zirconate titanate layer, a lanthanum strontium manganite layer, or any combination thereof.

10. The DNA sensing device of claim 2, wherein a first terminal of the negative capacitance layer is in electrical contact with the first subchamber and a second terminal of the negative capacitance layer is in electrical contact with the second subchamber.

11. The DNA sensing device of claim 10, wherein the first terminal is formed of a portion of a first surface of the negative capacitance layer that is not in contact with the hydrophobic layer.

12. The DNA sensing device of claim 10, wherein the second terminal is formed of a portion of a second surface of the negative capacitance layer, the second surface being an opposing surface of the first surface.

13. The DNA sensing device of claim 10, wherein the first terminal includes a hydrophobic material layer and the second terminal includes an adhesion/diffusion layer, the negative capacitance layer further comprising one or more of a strontium titanate layer, a strontium ruthenate layer, a barium titanate layer, a lead zirconate titanate layer, a lanthanum strontium manganite layer, or any combination thereof.

14. A method of fabricating a DNA sensing device, comprising:
providing a first electrode;
providing a second electrode;
providing a hydrophobic layer;
disposing a nanopore in the hydrophobic layer; and
providing a negative capacitance layer.

15. The method of claim 14, further comprising providing a chamber divided into a first subchamber and a second subchamber.

16. The method of claim 14, wherein providing the hydrophobic layer further includes dividing the chamber into a first subchamber and a second subchamber.

17. The method of claim 14, wherein disposing the nanopore in the hydrophobic layer includes disposing the nanopore such that the first subchamber and the second subchamber are in fluid communication with one another via the nanopore.

18. The method of claim 15, wherein:
providing the first electrode includes disposing the first electrode in contact with the first subchamber; and
providing the second electrode includes disposing the second electrode in contact with the second subchamber.

19. The method of claim 14, further comprising providing a voltage source that is electrically coupled to the first electrode and the second electrode.

20. The method of claim 14, wherein providing the negative capacitance layer includes disposing a first terminal of the negative capacitance layer in electrical contact with the first electrode and a second terminal of the negative capacitance layer in electrical contact with the second electrode.

21. The method of claim 20, further comprising providing a via configured to electrically couple the first terminal of the negative capacitance layer to the first electrode and the second terminal of the negative capacitance layer to the second electrode; wherein
providing the negative capacitance layer includes disposing the negative capacitance layer in the via.

22. The method of claim 20, wherein the first terminal includes a first adhesion/diffusion layer and the second terminal includes a second adhesion/diffusion layer, the negative capacitance layer further comprising a strontium titanate layer, a strontium ruthenate layer, a barium titanate layer, a lead zirconate titanate layer, a lanthanum strontium manganite layer, or any combination thereof.

23. The method of claim 15, wherein providing the negative capacitance layer includes disposing a first terminal of the negative capacitance layer in electrical contact with the first subchamber and disposing a second terminal of the negative capacitance layer in electrical contact with the second subchamber.

24. The method of claim 23, wherein the first terminal is formed of a portion of a first surface of the negative capacitance layer that is not in contact with the hydrophobic layer.

25. The method of claim 23, wherein the second terminal is formed of a portion of a second surface of the negative capacitance layer, the second surface being an opposing surface of the first surface.

26. The method of claim 18, wherein the first terminal includes a hydrophobic material layer and the second terminal includes an adhesion/diffusion layer, the negative capacitance layer further comprising one or more of a strontium titanate layer, a strontium ruthenate layer, a barium titanate layer, a lead zirconate titanate layer, a lanthanum strontium manganite layer, or any combination thereof.

27. A DNA sensing device, comprising:
a first electrode;
a second electrode;
a hydrophobic layer having a nanopore disposed therein; and
means for causing negative capacitance.

28. The DNA sensing device of claim 27, wherein means for causing negative capacitance is in electrical contact with the first electrode and the second electrode.

29. The DNA sensing device of claim 27, wherein means for causing negative capacitance is in electrical contact with the first subchamber and the second subchamber.

30. The DNA sensing device of claim 27, wherein a first terminal of means for causing negative capacitance includes a hydrophobic material layer and a second terminal of means for causing negative capacitance includes an adhesion/diffusion layer, means for causing negative capacitance further comprising one or more of a strontium titanate layer, a strontium ruthenate layer, a barium titanate layer, a lead zirconate titanate layer, a lanthanum strontium manganite layer, or any combination thereof.
